(19) United States
(12) Patent Application Publication
(10) Pub. No.: US 2010/0152826 A1
(43) Pub. Date: Jun. 17, 2010

(54) MYOCARDIAL ELECTRODE

(75) Inventors: Hiroaki Tanabe, Minato-ku (JP); Shuro Hayashi, Hiroshima-shi (JP)

Correspondence Address: ROBERTS MLOTKOWSKI SAFRAN & COLE, P.C.;Intellectual Property Department, P.O. Box 10064, MCLEAN, VA, 22102-8064, US

(73) Assignee: JMS CO., LTD., Hiroshima-shi, Hiroshima (JP)

(21) Appl. No.: 12/088,082

(22) PCT Filed: Sep. 25, 2006
(86) PCT No.: PCT/JP2006/318946

§ 371 Date: Mar. 25, 2008

(30) Foreign Application Priority Data
Sep. 26, 2005 (JP) 2005-277635

(51) Int. Cl. A61N 1/05

(52) U.S. Cl. 607/129

(57) ABSTRACT

Two through holes are formed in a first electrode part 10 to penetrate the first electrode part 10 in the extending direction of a substrate 30. The same two through holes are also formed in a second electrode part 20. A first lead 40 is inserted in and connected to one of the through holes of the first electrode part 10. A second lead 50 is inserted in the other through hole of the first electrode part 10 while it is insulated from the first electrode part 10 and engaged with an opening edge of the other through hole. An end of the second lead 50 is inserted in and connected to one of the through holes of the second electrode part 20. An interval between the first electrode part 10 and the second electrode part 20 is determined by the second lead 50.

### TECHNICAL FIELD

In a cardiac surgery involving thoracotomy, such as a coronary artery bypass surgery, arrhythmia caused by ventricular and atrial fibrillation is likely to occur. Therefore, electrical stimulation is given to cardiac muscle for defibrillation. In such a case, for example, a myocardial electrode disclosed by Patent Literature 1 may be used. The myocardial electrode of Patent Literature 1 includes two leads connected to positive and negative electrodes of an external cardiac pacemaker, two electrode parts connected to the distal ends of the leads and a substrate supporting the electrode parts at an interval from each other. The substrate is made of insulating material such as silicone rubber and embedded portions of the two electrode parts are embedded in the substrate.

When the cardiac pacemaker is actuated with the conductive surfaces of the electrode parts exposed on the substrate in contact with the surface of the heart, current from the cardiac pacemaker flows into the cardiac muscle through the leads and the electrode parts. As a result, electrical stimulation is given to the cardiac muscle and the defibrillation is carried out.

The myocardial electrode is also used to give the electrical stimulation to the cardiac muscle when cardiac motion deteriorates during a beating heart surgery without using a pump-oxygenator such that the rhythm of the cardiac motion returns to and remains normal. Further, the myocardial electrode is also used in an arrested heart surgery using the pump oxygenerator to restart the heartbeat by giving the electrical stimulation to the cardiac muscle.

For the manufacture of the myocardial electrode, the substrate in which the embedded portions the two electrode parts are embedded is formed by so-called insert molding, Specifically, the two electrode parts are placed in a cavity of a substrate mold and then a substrate material such as a liquid resin is poured in the cavity and cured.

[Patent Literature 1] Japanese Unexamined Patent Publication No. 2004-89384

### DISCLOSURE OF THE INVENTION

### Problem that the Invention is to Solve

In the manufacture of the substrate in which the embedded portions of the two electrode parts are embedded in the manner disclosed by Patent Literature 1, the two electrode parts placed in the cavity of the mold may be misaligned from the predetermined positions by the flow of the resin poured into the cavity. If the substrate material is cured with the electrode parts misaligned from the desired positions, the interval between the two electrode parts of the myocardial electrode may become too small or too large as compared with the predetermined interval. In such a case, desired electrical stimulation cannot be given to the heart. Thus, effective defibrillation cannot be performed and the return to the normal rhythm and the restart of the heartbeat of the arrested heart become difficult.

As a solution to this, the two electrode parts are kept still in the substrate mold to prevent the misalignment from the predetermined positions. In this case, an additional structure for keeping the electrode parts still has to be provided in the substrate mold. Therefore, the configuration of the substrate mold is complicated and the cost of the mold increases. This leads to an increase of the cost of the myocardial electrode.

Under these circumstances, the present invention has been achieved. In the manufacture of the substrate in which the embedded portions of the electrode parts are embedded, an object of the invention is to make it possible to determine the interval between the electrode parts using the existing leads connecting the cardiac pacemaker and the electrode parts such that the predetermined interval is kept between the electrode parts without complicating the configuration of the substrate mold and the cost of the myocardial electrode is reduced.

### Means of Solving the Problem

According to the present invention, the object is achieved by fixing a lead connected to a second electrode part while it is insulated from a first electrode part and determining the interval between the first and second electrode parts by the lead.

Specifically, according to a first aspect of the invention, a myocardial electrode includes: a first electrode part and a second electrode part; a first lead and a second lead for connecting the first electrode part and the second electrode part to a cardiac pacemaker, respectively; and an insulating substrate in which a first embedded portion of the first electrode part and a second embedded portion of the second electrode part are embedded, wherein the first electrode part and the second electrode part are arranged at an interval from each other while conductive surfaces thereof are exposed on the substrate and part of the second lead close to the electrode part is connected to the second embedded portion and fixed to and insulated from the first embedded portion and the interval between the first electrode part and second electrode part is determined by the part of the second lead close to the electrode part.

With this configuration, the second lead is connected to the second embedded portion and integrated with the second electrode part, while the second lead is fixed to and insulated from the first embedded portion and integrated with the first electrode part. The interval between the first and second electrode parts is determined by the second lead. Thus, a predetermined interval is kept between the first and second electrode parts by the second lead.

In the manufacture of the substrate, the first and second electrode parts and part of the second lead close to the electrode part are placed in a cavity of a mold for forming the substrate. Then, a substrate material in a liquid state is poured into the cavity. At this time, as the predetermined interval is kept between the first and second electrode parts by the second lead, the interval between the electrode parts does not become too small or too large even if the flow of the material poured into the cavity hits the first and second electrode parts.

According to a second aspect of the invention related to the first aspect, the conductive surfaces of the first electrode part and the second electrode part are exposed only on one surface of the substrate.

With this configuration, the one surface of the substrate is faced toward the heart such that the conductive surfaces of the first and second electrode parts are brought into contact with the heart. The conductive surfaces of the electrode parts do not come into contact with the other body tissue to which the electrical stimulation is unnecessary. Thus, the current of the cardiac pacemaker is allowed to flow into the cardiac muscle only and prevented from flowing into the other body tissue.

According to a third aspect of the invention related to the first aspect, the substrate includes an electrode part support in which the first embedded portion and the second embedded portion are embedded and an extension part extending from the electrode part support to be in contact with body tissue.

With this configuration, when the conductive surfaces of the first and second electrode parts are brought into contact with the heart, the extension part extended from the electrode part support comes into contact with the heart and the body tissue around the heart. Therefore, the electrode part support is stabilized and the conductive surfaces are prevented from coming off the surface of the heart.

According to a fourth aspect of the invention related to the third aspect, a separation guide at which the extension part is separated from the electrode part support is provided between the electrode part support and the extension part of the substrate,

With this configuration, if the extension part of the substrate is unnecessary, it can easily be separated from the electrode part support to downsize the substrate. Therefore, the first and second electrode parts can be placed in a small area in the thoracic cavity.

According to a fifth aspect of the invention related to the fourth aspect, the electrode part support of the substrate is configured to be insertable into a drain for discharging body fluid seeped into a thoracic cavity to the outside.

With this configuration, when the electrode parts and the substrate are left in the thoracic cavity during a follow-up after a cardiac surgery involving thoracotomy and then the myocardial electrode is no longer necessary, the electrode parts and the substrate are taken out of the thoracic cavity through the drain.

According to a sixth aspect of the invention related to the first aspect, the fast embedded portion and the second embedded portion are formed to extend in the extending direction of the substrate.

With this configuration, the first and second embedded portions extend in the extending direction of the substrate. Therefore, the first and second electrode parts are supported on the substrate with stability.

According to a seventh aspect of the invention related to the first aspect, a through hole is formed in the first embedded portion to penetrate the first embedded portion in the extending direction of the substrate and the part of the second lead close to the electrode part is inserted in the through hole and engaged with an opening edge of the through hole to be fixed to the first embedded portion.

With this configuration, the second lead is fixed to the first embedded portion by passing the second lead through the first embedded portion.

### EFFECT OF THE INVENTION

According to the first aspect of the invention, the second lead is connected to the second embedded portion of the second electrode part and fixed to and insulated from the first embedded portion such that the interval between the electrode parts is determined by the second lead. Therefore, the predetermined interval is kept between the first and second electrode parts without complicating the configuration of the mold for forming the substrate in which the first and second embedded portions are embedded. Thus, the mold of the substrate becomes less expensive and the cost is reduced.

According to the second aspect of the invention, the conductive surfaces of the first and second electrode parts are exposed only on one surface of the substrate. Therefore, only the cardiac muscle is electrically stimulated, while the other body tissue to which the electrical stimulation is unnecessary is not electrically stimulated. Thus, the therapy is carried out less invasively.

According to the third aspect of the invention, the extension part extending from the electrode part support is brought into contact with the body tissue to stabilize the electrode part support. Therefore, the conductive surfaces are prevented from coming off the surface of the heart and the electrical stimulation is surely given to the cardiac muscle.

According to the fourth aspect of the invention, the separation guide is provided between the electrode part support and the extension part. This makes it possible to easily separate the extension part from the electrode part support to downsize the substrate. Therefore, the electrode parts are easily and quickly placed in a small area in the thoracic cavity.

According to the fifth aspect of the invention, the electrode parts and the substrate left in the thoracic cavity are taken out of the thoracic cavity through the drain for discharging body fluid from the thoracic cavity without making a thoracoabdominal incision. Therefore, the therapy is carried out less invasively.

According to the sixth aspect of the invention, the first and second embedded portions extend in the extending direction of the substrate. Therefore, the first and second electrode parts are supported on the substrate with stability and the electrical stimulation is surely given to the cardiac muscle.

According to the seventh aspect of the invention, the second lead is inserted in the through hole of the first embedded portion and engaged with the opening edge of the through hole to be fixed to the first embedded portion. Thus, the second lead is arranged within the first embedded portion and the first electrode part and the second lead are made compact.

### BRIEF DESCRIPTION OF DRAWINGS

FIGS. 1A to 1C illustrate a myocardial electrode of the present invention. FIG. 1A is a plan view, FIG. 1B is a side view and FIG. 1C is a view observed in the direction of an arrow Y.

FIGS. 2A and 2B illustrate a first electrode part. FIG. 2A is a plan view and FIG. 2B is a side view observed from the side on which through holes are opened.

FIG. 3 is a plan view illustrating a first lead and a second lead.

FIG. 4 is a plan view illustrating the first lead and the second lead connected to a first electrode part and a second electrode part, respectively.

FIG. 5 is a view illustrating how the myocardial electrode is used in a coronary artery bypass surgery on a beating heart.

FIG. 6 is a view illustrating how the myocardial electrode is used after the coronary artery bypass surgery.

FIG. 7 is a plan view illustrating the substrate protruding from an end of a first drain.

FIG. 8 is a view corresponding to FIG. 7 illustrating a first modification of the embodiment.

FIG. 9 is a view corresponding to FIG. 1A illustrating a second modification of the embodiment.

FIGS. 10A to 10C are views corresponding to FIGS. 1A to 1C illustrating a third modification of the embodiment.

FIG. 11 is a view corresponding to FIG. 6 illustrating a third modification of the embodiment.

FIGS. 12A to 12C are views corresponding to FIGS. 1A to 1C illustrating a fourth modification of the embodiment.

FIG. 13 is a view corresponding to FIG. 1B illustrating a fifth modification of the embodiment.

### EXPLANATION OF REFERENCE NUMERALS

1 Myocardial electrode

10 First electrode part

14 Through hole

15 First embedded portion

20 Second electrode part

25 Second embedded portion

30 Substrate

31 Electrode part support

32 Extension part

34 Groove (separation guide)

40 First lead

50 Second lead

60 First drain

P Cardiac pacemaker

### BEST MODE FOR CARRYING OUT THE INVENTION

Hereinafter, embodiments of the present invention will be described with reference to the drawings. The following embodiments are given for illustrative purpose only and do not intend to limit the present invention, application range and use thereof.

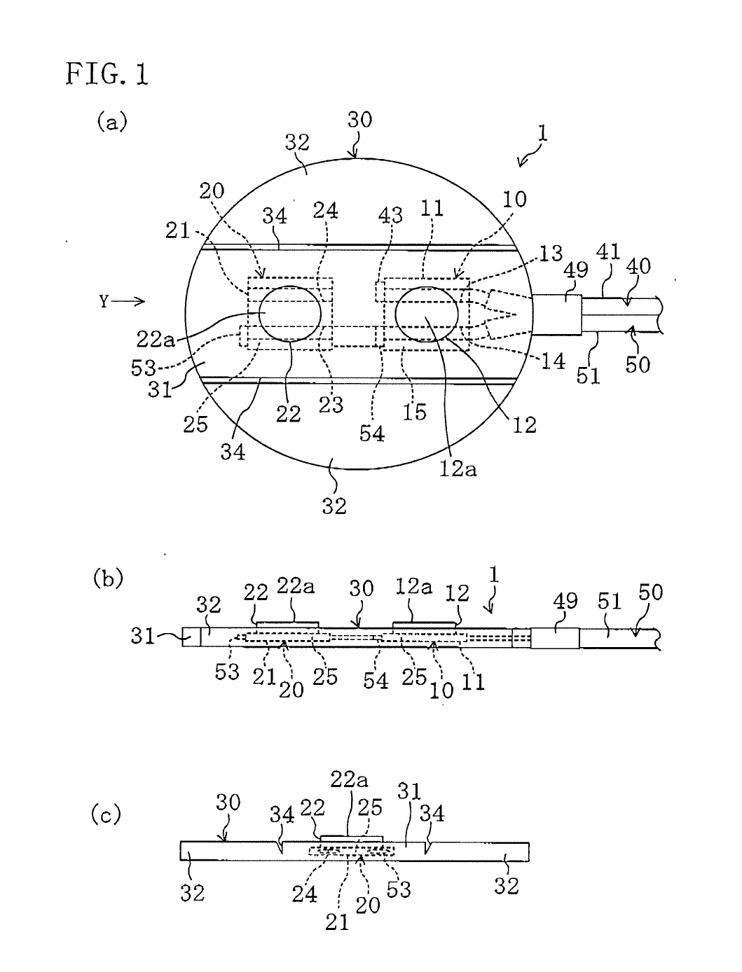

FIGS. 1A to 1C show a myocardial electrode 1 of an embodiment of the present invention. The myocardial electrode 1 is used, for example, to give electrical stimulation to cardiac muscle to stop ventricular and atrial fibrillation which occurs during or after a coronary artery bypass surgery. The myocardial electrode 1 is also used to give electrical stimulation to the cardiac muscle when cardiac motion deteriorates during a beating heart surgery without using a pump-oxygenator (not shown) such that the rhythm of the cardiac motion returns to and remains normal. Further, the myocardial electrode 1 is also used in an arrested heart surgery using the pump oxygenerator to restart the heartbeat by giving the electrical stimulation to the cardiac muscle.

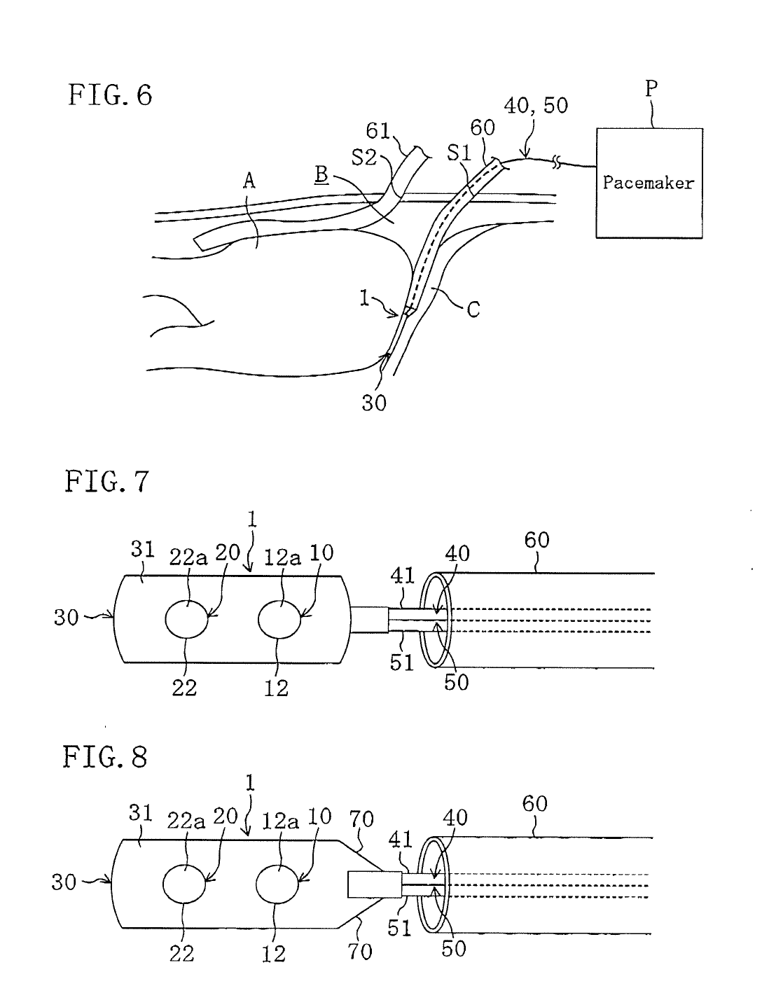

The myocardial electrode 1 includes a first electrode part 10, a second electrode part 20, a substrate 30 for supporting the first and second electrode parts 10 and 20 at an interval from each other and first and second leads 40 and 50 for connecting the first and second electrode parts 10 and 20 to a cardiac pacemaker P (shown in FIGS. 5 and 6), respectively. The cardiac pacemaker P to which the myocardial electrode 1 is connected is an external pacemaker which has widely been used in the medical field and configured to apply a desired pulse current.

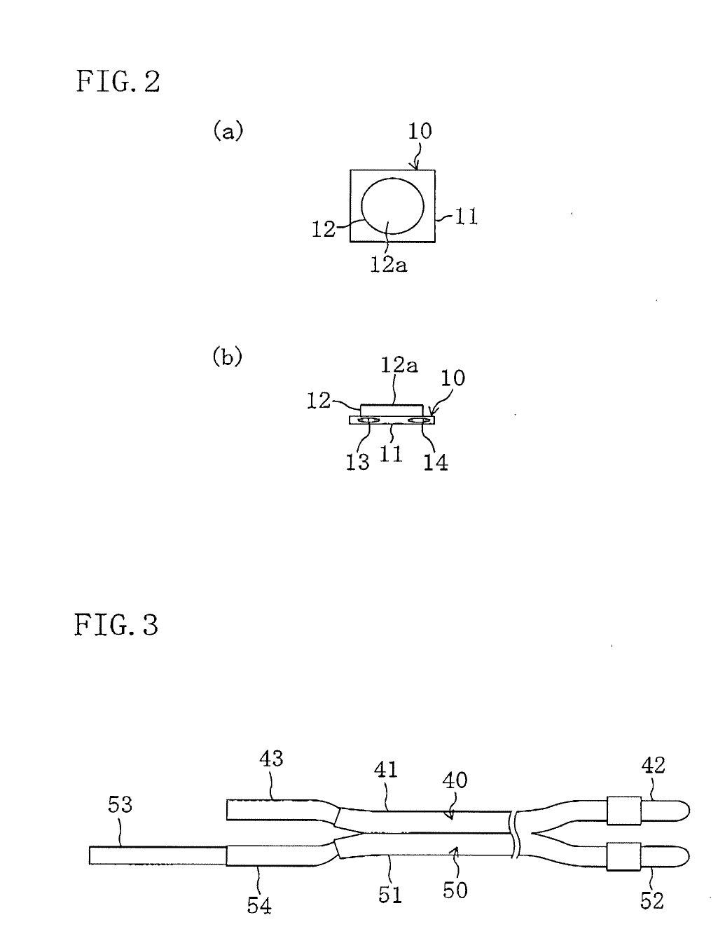

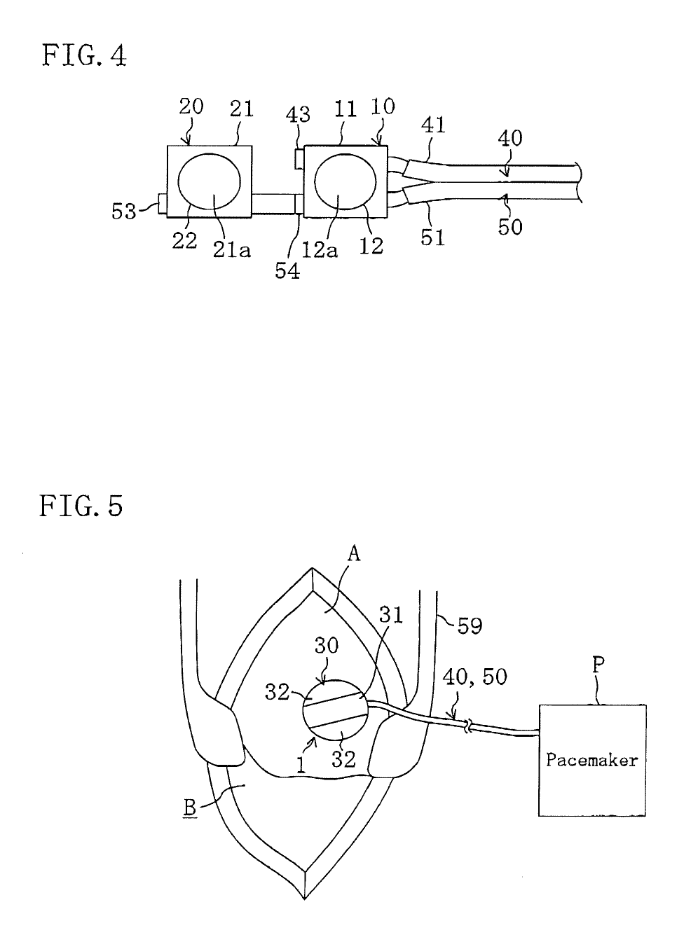

As shown in FIGS. 2A and 2B, the first electrode part 10 includes a plate-like rectangular part 11 and a plate-like round part 12 protruding from one surface of the rectangular part 11. The rectangular part 11 and the round part 12 are integrated into one piece. The thickness of the rectangular part 11 is smaller than that of the substrate 30 as shown in FIGS. 1B and 1C. The rectangular part 11 and a portion of the round part 12 close to the rectangular part 11 are embedded in the substrate 30 as a first embedded portion 15. The rectangular part 11 embedded in the substrate 30 is arranged to extend in the extending direction of the substrate 30.

In the rectangular part 11, two through holes 13 and 14 extending in the extending direction of the rectangular part 11 are formed at an interval from each other to have openings on the vertical sides of the rectangular part 11. The through holes 13 and 14 have the same shape and their inner diameter is determined such that parts of the first and second leads 40 and 50 close to the electᵣode ₚarts can be inserted therein.

The external size of the round part 12 is set smaller than that of the rectangular part 11. The round part 12 is located substantially on the center of the one surface of the rectangular part 11. Therefore, when the first electrode part 10 is viewed in plan, the rectangular part 11 protrudes from the circumference of the round part 12. As the rectangular part 11 is formed larger than the round part 12 and configured to extend in the extending direction of the substrate 30, the first electrode part 10 is supported on the substrate 30 with stability.

With the first embedded portion 15 embedded in the substrate 30, a portion of the round part 12 opposite the rectangular part 11 is exposed and protrudes from only the one surface of the substrate 30. The portion of the round part 12 protruding from the substrate 30 serves as a conductive surface 12a. The second electrode part 20 is configured in the same manner as the first electrode part 10. That is, it includes a rectangular part 21 having two through holes 23 and 24 and a round part 22 including a conductive surface 22a. The rectangular part 21 and a portion of the round part 22 close to the rectangular part 21 are embedded in the substrate 30 as a second embedded portion 25.

As shown in FIG. 3, the first lead 40 is prepared by covering a conductor formed of a strand of thin steel wires with a coating 41 such as a resin. A connection part 42 (shown in FIG. 3 only) for connection to a positive terminal of the cardiac pacemaker P is provided at an end of the first lead 40. The other end of the first lead 40 close to the electrode part is embedded in the substrate 30 together with the rectangular part 11 of the first electrode part 10 as shown in FIG. 1. A cylindrical metallic cover 43 for covering the conductor is fixed onto the end of the first lead 40 close to the electrode part. The dimension of the cover 43 in the direction of the center line is set longer than that of the through holes 13 and 14 of the first electrode part 10. The end of the first lead 40 close to the electrode part is inserted in the through hole 13 of the first electrode part 10 with the cover 43 fixed thereon. The first electrode part 10 is electrically connected to the conductor via the cover 43.

With the end of the first lead 40 inserted in the through hole 13, the ends of the cover 43 in the direction of the center line protrude from the openings of the through hole 13. Part of the cover 43 protruding from the through hole 13 is crushed such that its width becomes larger than that of the opening of the through hole 13. Therefore, the cover 43 is engaged with an opening edge of the through hole 13. As a result, the end of the first lead 40 close to the electrode part is prevented from coming off the through hole 13.

Just like the first lead 40, the second lead 50 is prepared by covering a conductor with a coating 51 and a connection part 52 for connection to a negative terminal of the cardiac pacemaker P is provided at an end thereof as shown in FIG. 3. The other end of the second lead 50 close to the electrode part extends longer than the end of the first lead 40 close to the electrode part and embedded in the substrate 30 together with the rectangular part 21 of the second electrode part 20. A cover 53 similar to the cover 43 of the first lead 40 is provided at the end of the second lead 50 close to the electrode part, With the end of the second lead 50 close to the electrode part inserted in the through hole 23 of the second electrode part 20, the cover 53 is engaged with an opening edge of the through hole 23. Reference numeral 49 in FIG. 1 indicates a lead cover made of a resin.

Part of the second lead 50 closer to the connection part 52 than to the cover 53 is covered with an insulating element 54 different from the coating 51. The outer diameter of the insulating element 54 is larger than the inner diameter of the through hole 14 of the first electrode part 10. When the part of the second lead 50 covered with the insulating element 54 is inserted in the through hole 14, the insulating element 54 is engaged with the opening edge of the through hole 14 such that the second lead 50 is fixed to the first electrode part 10. Specifically, the first and second electrode parts 10 and 20 are integrated into one piece by the second lead 50 and the interval between the first and second electrode parts 10 and 20 is determined by optionally adjusting the positions of the electrode parts 10 and 20 with respect to the second lead 50. The through hole 24 of the second electrode part 20 is opened at both ends.

The substrate 30 is made of an insulating material such as silicone rubber and disc-shaped as shown in FIGS. 1A to 1C. The substrate 30 includes a narrow electrode part support 31 extending along the diameter of the substrate 30 to pass the center of the substrate 30 and two extension parts 32 extending outward from the lengthwise sides of the electrode part support 31, respectively. The first embedded portion 15 and the second embedded portion 25 are embedded in the electrode part support 31 at an interval from each other in the lengthwise direction of the electrode part support 31. A groove 34 is provided between the electrode part support 31 and each of the extension parts 32. The groove 34 has a substantially V-shaped cross section which is opened on one side of the substrate 30 as shown in FIG. 1C and functions as a separation guide at which the extension parts 32 are separated from the electrode part support 31.

Now, the manufacture of the thus-configured myocardial electrode 1 will be described. First, part of the first lead 40 close to the electrode ₚart is inserted in the through hole 13 of the first electrode part 10 and fixed to the first electrode part 10. Part of the second lead 50 close to the electrode part is inserted in the through hole 14 of the first electrode part 10 and part of the second lead 50 covered with the insulating element 54 is placed in the through hole 14 and engaged with the opening edge of the through hole 14. As a result, the second lead 50 is fixed to and insulated from the first embedded portion 15. Then, part of the second lead 40 close to the electrode part is inserted in the through hole 23 of the second electrode part 20 to be fixed to the second electrode ₚart 20. The interval between the first and second electrode parts 10 and 20 is determined such that desired electrical stimulation is given to the cardiac muscle.

Then, the first and second electrode parts 10 and 20 are placed in a cavity of a mold (not shown) for forming the substrate 30 together with the parts of the first and second leads 40 and 50 close to the electrode part. After the mold is closed, a material of the substrate 30 in a liquid state is poured into the cavity. Then, the flow of the material hits the first and second electrode parts 10 and 20. As the predetermined interval is kept between the first and second electrode parts 10 and 20 by the second lead 50, the interval between the first and second electrode parts 10 and 20 is prevented from becoming too small or too large, After the material in the cavity is cured, the mold is opened and the substrate 30 is released from the mold. Thus, the myocardial electrode 1 including the substrate 30 in which the embedded portions 15 and 25 of the first and second electrode parts 10 and 20 are embedded is provided by insert molding.

Use of the myocardial electrode 1 in a coronary artery bypass surgery on a beating heart will be described with reference to FIG. 5. Reference numeral 59 in FIG. 5 indicates a rib spreader for keeping an incision in the thorax open. First, as the substrate 30 is placed on a heart A with one surface facing the heart A, the conductive surfaces 12a and 22a come into contact with the heart A. At the same time, the extension parts 32 extending from the electrode part support 31 of the substrate 30 are also in contact with the heart A. Therefore, the electrode part support 31 remains stable while the heart is beating and the conductive surfaces 12a and 22a are prevented from coming off the heart A.

When the cardiac pacemaker P is actuated, current from the cardiac pacemaker P flows into the first and second electrode parts 10 and 20 through the first and second leads 40 and 50, respectively, and electrical stimulation is given to the cardiac muscle by the first and second electrode parts 10 and 20. Since the predetermined interval is kept between the first and second electrode parts 10 and 20 as described above, desired electrical stimulation is given to the cardiac muscle and defibrillation is effectively performed.

Further, as the conductive surfaces 12a and 22a of the first and second electrode parts 10 and 20 are exposed only on the one surface of the substrate 30, the conductive surfaces 12a and 22a are brought into contact with only a required part of the heart A and do not come in contact with other body tissue to which the electrical stimulation is unnecessary. Thus, the current of the cardiac pacemaker P is allowed to flow into the cardiac muscle only and prevented from flowing into the other body tissue.

The first and second electrode parts 10 and 20 are easily placed in a small area in a thoracic cavity B by downsizing the substrate 30 by separating the extension parts 32 from the electrode part support 31. In this case, both or one of the two extension parts 32 may be separated from the electrode part support 31.

Next, use of the myocardial electrode 1 after the coronary artery bypass surgery will be described with reference to FIGS. 6 and 7. After the coronary artery bypass surgery, body fluid seeped into the thoracic cavity B is discharged out of the thoracic cavity B using a first drain 60 and a second drain 61. The drains 60 and 61 are circular tubes made of a resin. The width of the electrode part support 31 of the substrate 30 is set smaller than the inner diameter of the drains 60 and 61 such that the electrode part support 31 can be inserted in the drains 60 and 61.

The first drain 60 is arranged such that an end thereof is inserted between the heart A and a diaphragm C and the other end protrudes out of the thoracic cavity B. The second drain 61 is arranged such that an end thereof is inserted in part of the thoracic cavity B above the heart A and the other end protrudes out of the thoracic cavity B.

In this case, as shown in FIG. 7, both of the extension parts 32 of the substrate 30 are separated from the electrode part support 31 and the remaining substrate 30 is inserted in the first drain 60 such that it protrudes from an end of the first drain 60. After the surgery, the first drain 60 and the substrate 30 is inserted between the heart A and the diaphragm C while the other end of the first drain 60 protrudes out of the thoracic cavity B through an incision Si. At the same time, the substrate 30 is sandwiched and fixed between the heart A and the diaphragm C with the conductive surfaces 12a and 22a facing the heart A. Thus, the conductive surfaces 12a and 22a are brought into contact with the heart A. The second drain 61 is also inserted in the thoracic cavity B at one end while it protrudes out of the thoracic cavity B through an incision S2 at the other end. Reservoir bags (not shown) for keeping the body fluid discharged from the thoracic cavity B are attached to the other ends of the first and second drains 60 and 61, respectively.

When the cardiac pacemaker P is actuated, current from the cardiac pacemaker P flows into the cardiac muscle. As the predetermined interval is kept between the first and second electrode parts 10 and 20, the defibrillation is effectively performed. Further, since the conductive surfaces 12a and 22a are exposed only on the one surface of the substrate 30, the electrical stimulation is not given to the diaphragm C.

When the condition of the patient becomes stable after the surgery, the first and second electrode parts 10 and 20 and the substrate 30 are taken out of the thoracic cavity B. For taking them out, the first and second leads 40 and 50 are pulled at the connection parts 42 and 52. Then, the substrate 30 is drawn into the first drain 60 from the first electrode parts 10. As the first and second leads 40 are 50 pulled more, the first and second electrode parts 10 and 20 and the substrate 30 pass through the first drain 60 and are taken out of the thoracic cavity B. Thus, there is no need of making a thoracoabdominal incision to take out the first and second electrode parts 10 and 20 and the substrate 30.

As described above, the myocardial electrode 1 of the present embodiment is configured such that the second lead 50 connected to the second electrode part 20 is fixed to the first electrode part 10 and the interval between the first and second electrode parts 10 and 20 is determined by the second lead 50. Therefore, in the manufacture of the substrate 30 in which the first and second embedded portions 15 and 25 are embedded, the predetermined interval is kept between the first and second electrode parts 10 and 20 without complicating the configuration of the mold. Thus, the mold of the substrate 30 becomes less expensive and the cost is reduced.

Since the conductive surfaces 122 and 22a of the first and second electrode parts 10 and 20 are exposed only on the one surface of the substrate 30, only the cardiac muscle is electrically stimulated, while the other body tissue to which the electrical stimulation is unnecessary is not electrically stimulated. Thus, the therapy is carried out less invasively.

The substrate 30 is provided with the electrode part support 31 and the extension parts 32 extending from the electrode part support 31 to be in contact with the body tissue. Therefore, when the conductive surfaces 12a and 22a of the first and second electrode parts 10 and 20 are brought into contact with the heart A, the extension parts 32 come into contact with the heart A and the body tissue around the heart A to stabilize the electrode part support 31. Accordingly, the conductive surfaces 12a and 22a are prevented from coming off the surface of the heart A and the electrical stimulation is surely given to the cardiac muscle.

With the provision of the grooves 34 between the electrode part support 31 and the extension parts 32 of the substrate 30, the extension parts 32 are easily separated from the electrode part support 31 to downsize the substrate 30. Therefore, the first and second electrode parts 10 and 20 can be inserted easily and quickly in a small area in the thoracic cavity B.

The electrode part support 31 of the substrate 30 is configured to be insertable in the first drain 60. Therefore, when the first and second electrode parts 10 and 20 are left in the thoracic cavity B during a follow-up after the surgery and then the electrical stimulation is no longer necessary, the first and second electrode parts 10 and 20 and the substrate 30 are taken out without making a thoracoabdominal incision. Thus, the therapy is carried out less-invasively.

The rectangular parts 11 and 21 are formed to extend in the extending direction of the substrate 30. Therefore, the first and second electrode parts 10 and 20 axe supported on the substrate 30 with stability.

The rectangular part 11 is provided with the through hole 14 penetrating the rectangular part 11 in the extending direction of the substrate 30 and part of the second lead 50 close to the electrode part is inserted in the through hole 14 and engaged with the opening edge of the through hole 14 to be fixed the first electrode part 10. Therefore, the second lead 50 is arranged within the rectangular part 11. Thus, the first electrode part 10 and the second lead 50 are made compact.

With use of a myocardial electrode of a needle type or an alligator clip type which has conventionally used in a cardiac surgery, the cardiac muscle may be scratched and damaged. In contrast, the myocardial electrode 1 of the present embodiment is used b_{y} merely applying the first and second electrode parts 10 and 20 onto the cardiac muscle. Therefore, the electrical stimulation is applied less invasively without causing any damage to the cardiac muscle.

According to a first modification of the embodiment shown in FIG. 8, an edge of the electrode part support 31 close to the first electrode part 10 may be provided with a guiding part 70 for guiding the substrate 30 into the first drain 60 when the substrate 30 is drawn into the first drain 60. The guiding part 70 is configured to protrude from the electrode part support 31 toward the connection parts 42 and 52 of the leads 40 and 50 and tapered toward the connection parts 42 and 52 of the leads 40 and 50 with respect to the center line of the leads 40 and 50. With this configuration, when the substrate 30 left in the thoracic cavity B is taken out through the first drain 60, the guiding part 70 slides along the opening edge of the first drain 60 such that the substrate 30 is smoothly drawn into the first drain 60.

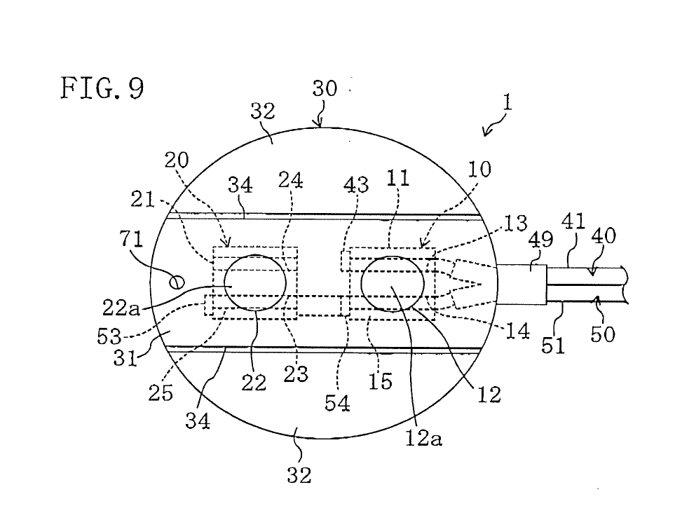

According to a second modification of the embodiment shown in FIG. 9, a hole 71 may be provided in the vicinity of the edge of the electrode part support 31 to penetrate the electrode part support 31 in the thickness direction. With this configuration, the electrode part support 31 placed between the heart A and the diaphragm C is fixed to that position by sewing the electrode part support 31 onto the surface tissue of the heart A with a thread run through the hole 71. Thus, the electrode part support 31 is kept at the predetermined position.

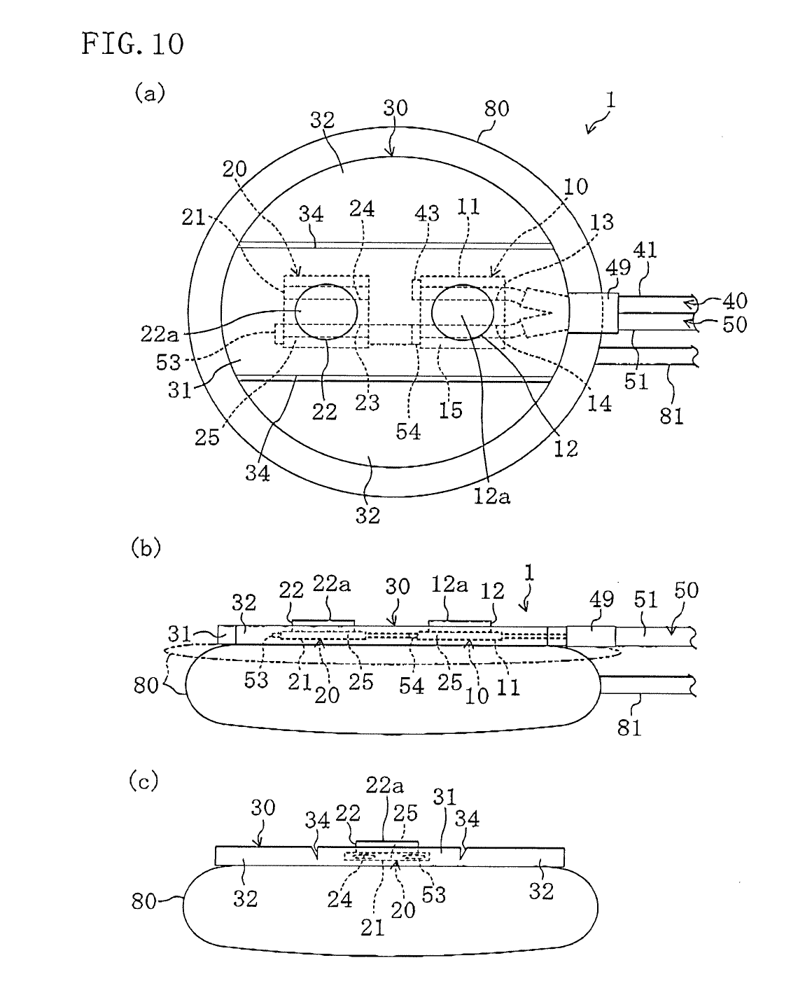

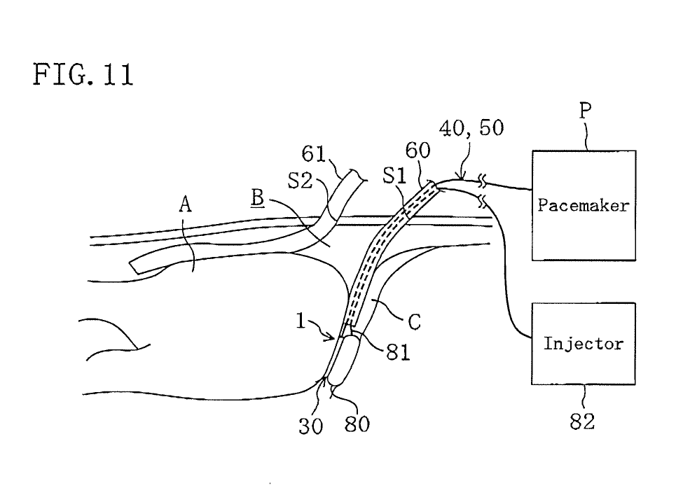

According to a third modification of the embodiment shown in FIGS. 10A to 10C and 11, a balloon 80 for pressing the electrode part support 31 against the surface of the heart A may be provided on the surface of the electrode part support 31 opposite the conductive surfaces 12a and 22a. The balloon 80 is circular and larger than the substrate 30 and adhered to the electrode part support 31 with an adhesive, for example. An end of an air line 81 is connected to the balloon 80 to communicate with the inner space of the balloon 80. The air line 81 is made of a flexible resin and has a diameter smaller than the inner diameter of the first drain 60. The other end of the air line 81 is connected to an air injector 82. The injector 82 may be a syringe, for example.

According to the third modification, the balloon 80 in a deflated state (indicated by a broken line in FIG. 10B). As shown in FIG. 11, the electrode part support 31 are placed between the heart A and the diaphragm C and then the balloon 80 is inflated by injecting air into the balloon 80 with the injector 82. As a result, the electrode part support 31 is brought into close contact with the heart A. Though not shown, after the balloon 80 is inflated, the air flow may be blocked by pinching the middle part of the air line 81 with a clip or closing a switching valve provided in the middle of the air line 81. For taking the electrode part support 31 out of the thoracic cavity B, the balloon 80 is deflated to shrink the balloon 80.

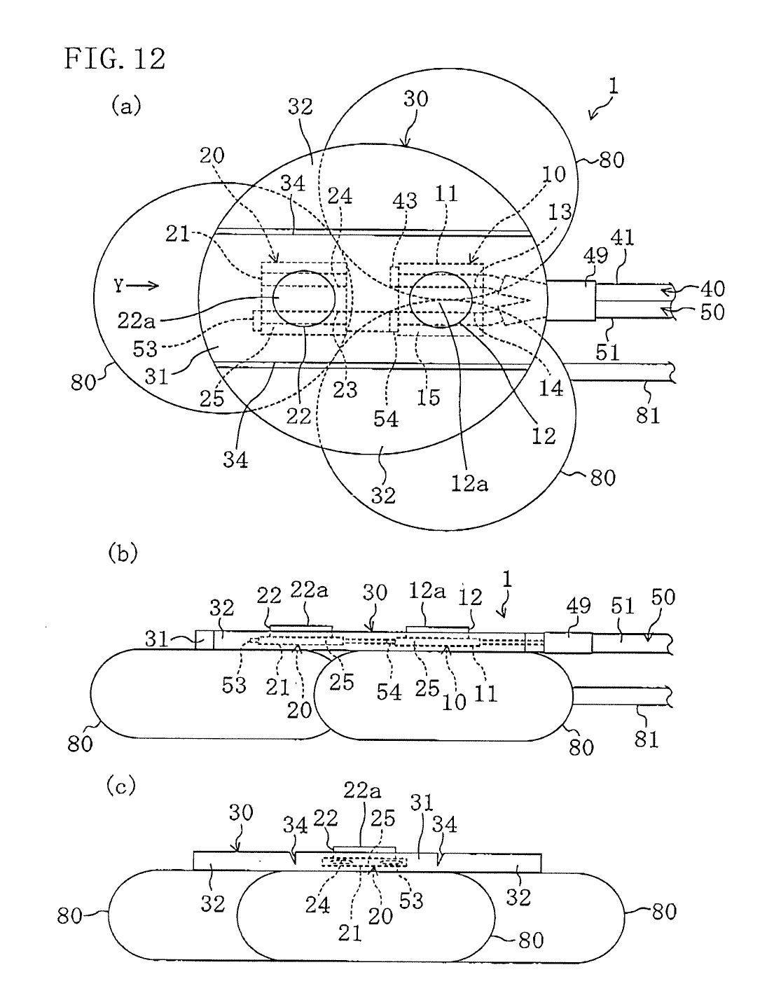

According to a fourth modification of the embodiment shown in FIGS. 12A to 12C, the electrode part support 31 may be provided with three balloons 80. In this modification, the inner spaces of the three balloons 80 are communicated with each other and the air line 81 is connected to one of the balloons 80. With the provision of the three balloons 80, the electrode part support 31 is brought into close contact with the heart A with stability. Thus, the electrical stimulation is surely given to the cardiac muscle.

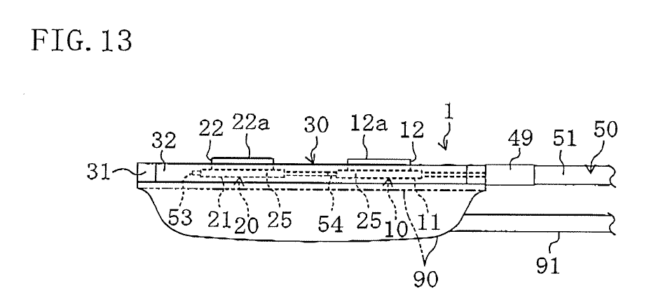

According to a fifth modification of the embodiment shown in FIG. 13, a thin circular sheet 90 made of silicone rubber may be used in place of the balloon 80. The periphery of the circular sheet 90 is adhered to the periphery of the substrate 30 with an adhesive and air is introduced between the sheet 90 and the substrate 30 to inflate the sheet 90. Reference numeral 91 indicates an air line similar to that used in the fourth modification and is connected to an injector.

The electrode part support 31 may be placed not only between the heart A and the diaphragm C but also between the heart A and the body tissue around the heart A such as a thoracic wall. Also in this case, the balloon 80 or the sheet 90 may be provided and inflated to keep the electrode part support 31 in close contact with the heart A.

The shapes of the first electrode part 10, the second electrode part 20 and the substrate 30 are not limited to those described above and may optionally be varied. Further, the material of the substrate 30 is not limited to silicone rubber and other kinds of rubbers and resins may be used.

The myocardial electrode 1 can be used also in other surgeries than the cardiac surgery, such as a surgery which requires thoracotomy and may cause fibrillation.

The separation guide provided between the electrode part support 31 and the extension parts 32 of the substrate 30 may be realized by other means than the grooves 34, such as a plurality of through holes or hollow parts.

### INDUSTRIAL APPLICABILITY

As described above, for example, the myocardial electrode of the present invention is used for stopping ventricular and atrial fibrillation which occurs during a coronary artery bypass surgery.

1. A myocardial electrode comprising:
a first electrode part and a second electrode part;
a first lead and a second lead for connecting the first electrode part and the second electrode part to a cardiac pacemaker, respectively; and
an insulating substrate in which a first embedded portion of the first electrode part and a second embedded portion of the second electrode part are embedded, wherein
the first electrode part and the second electrode part are arranged at an interval from each other while conductive surfaces thereof are exposed on the substrate and
part of the second lead close to the electrode part is connected to the second embedded portion and fixed to and insulated from the first embedded portion and the interval between the first electrode part and second electrode part is determined by the part of the second lead close to the electrode part.

2. The myocardial electrode of claim 1, wherein
the conductive surfaces of the first electrode part and the second electrode part are exposed only on one surface of the substrate.

3. The myocardial electrode of claim 1, wherein
the substrate includes an electrode part support in which the first embedded portion and the second embedded portion are embedded and an extension part extending from the electrode part support to be in contact with body tissue.

4. The myocardial electrode of claim 3, wherein
a separation guide at which the extension part is separated from the electrode part support is provided between the electrode part support and the extension part of the substrate.

5. The myocardial electrode of claim 4, wherein
the electrode part support of the substrate is configured to be insertable into a drain for discharging body fluid seeped into a thoracic cavity to the outside.

6. The myocardial electrode of claim 1, wherein
the first embedded portion and the second embedded portion are formed to extend in the extending direction of the substrate.

7. The myocardial electrode of claim 1, wherein
a through hole is formed in the first embedded portion to penetrate the first embedded portion in the extending direction of the substrate and
the part of the second lead close to the electrode part is inserted in the through hole and engaged with an opening edge of the through hole to be fixed to the first embedded portion.
